(19) United States
(12) Patent Application Publication
(10) Pub. No.: US 2026/0014295 A1
(43) Pub. Date: Jan. 15, 2026

(54) COMPOSITION FOR TISSUE REPAIR COMPRISING COPOLYMER OF HYDROPHILIC BIOCOMPATIBLE POLYMER AND HYDROPHOBIC BIOCOMPATIBLE POLYMER

(71) Applicant: Rose Lab Co., Ltd., Seoul (KR)

(72) Inventors: Woo Sung Lee, Gyeonggi-do (KR); Hyeon Jin Jeon, Seoul (KR)

(21) Appl. No.: 18/869,138

(22) PCT Filed: Mar. 24, 2023
(86) PCT No.: PCT/KR2023/003957

§ 371 Date: Sep. 10, 2025

(30) Foreign Application Priority Data
May 25, 2022 (KR) 10-2022-0064347

(51) Int. Cl. A61L 27/26; A61L 27/18; A61L 27/58

(52) U.S. Cl. CPC A61L 27/26; A61L 27/18; A61L 27/58; A61L 2400/06; A61L 2430/34

(57) ABSTRACT

The present invention relates to a composition for tissue repair, the composition comprising a copolymer of a hydrophilic biocompatible polymer and a hydrophobic biocompatible polymer. The composition can be used as a composition for tissue repair due to being rapidly biodegradable in the body and having excellent biocompatibility and collagen regeneration effects, and can also be used as an injectable agent due to having the excellent property of being injectable into the body.

### TECHNICAL FIELD

The present invention relates to a composition for tissue repair, the composition comprising a copolymer of a hydrophilic biocompatible polymer and a hydrophobic biocompatible polymer. Specifically, the composition of the present invention can be used as an excellent injectable agent for tissue repair due to having thermosensitivity, being injectable into the body, and having collagen regeneration effects.

### BACKGROUND ART

Most people have wrinkles due to a lack of collagen production ability caused by aging, and when skin inflammation is severe, the dermis is deformed due to insufficient collagen production by fibroblasts, such as loss of dermal tissue, resulting in scars that are punctured with an awl or sunken in shape.

In cases where collagen production is insufficient due to loss of dermal tissue, wrinkles or scars often remain. In order to improve wrinkles or scars caused by such loss of dermal tissue, an injectable agent for tissue repair can be used, and a biocompatible injectable agent for tissue repair is used to restore dermal tissue.

Currently, as an injectable agent for tissue repair, products are mainly developed using hyaluronic acid gels for facial plastic surgery, and are widely used because they have excellent biocompatibility and are reabsorbed in the body. However, hyaluronic acid gels do not have the function of stimulating collagen production in dermal tissue, have large particle sizes, and have high viscosity. Due to these characteristics, direct injection into narrow and deep skin scars in the dermis may cause the surrounding skin to bulge, making them unsuitable. In addition, if side effects such as filler clumping occur, hyaluronic acid decomposition treatment must be performed by injecting hyaluronidase into the filler injection area to break down the existing hyaluronic acid, and severe bruising or swelling may occur in the filler injection area.

Meanwhile, injectable agents for tissue repair based on the existing synthetic polymers for facial plastic surgery, polycaprolactone or polylactic acid are recommended to be injected subcutaneously into deep wrinkles due to concerns about side effects such as clumping and non-inflammatory nodules when administered, and there is a problem that they must be surgically removed if side effects occur. In addition, the existing injectable agents for tissue repair based on polycaprolactone or polylactic acid have a characteristic of remaining in the body for a period of 12 to 24 months of biodegradability. Due to this characteristic, when the polycaprolactone or polylactic acid is applied subcutaneously, it exhibits a continuous tissue repair effect, but when applied intradermally, there is a problem that granulomas occur.

Regarding the above polycaprolactone-based composition or injectable agent for tissue repair, Korean Patent Application Publication No. 10-2020-0158100 presents a composition for tissue repair prepared as microparticles of a polymer of a hydrophilic biocompatible polymer and a hydrophobic biocompatible polymer, but reports on a formulation whose viscosity increases as the temperature increases. Korean Patent Application Publication No. 10-2015-0053181 presents a thermosensitive composition for tissue repair prepared as microparticles of a polymer of a hydrophilic biocompatible polymer and a hydrophobic biocompatible polymer. However, this has the characteristic of being in a gel form below 30° C. but in a sol form above 30° C. due to an increase in viscosity, so it is not suitable when injected into the skin in areas where dermis is lost as there is a high risk of side effects such as formation of nodules or filler clumps. In addition, Korean Patent Application Publication No. 10-2016-0122111 presents a composition for tissue repair prepared as microparticles of a polymer of a hydrophilic biocompatible polymer and a hydrophobic biocompatible polymer, but only reports the form existing as a suspension at 20 to 35° C., and no report is made on the change in properties according to the temperature of use. Korean Patent Application Publication No. 10-2015-0010464 presents a composition for tissue repair prepared as microparticles of a copolymer of a hydrophilic biocompatible polymer and a hydrophobic biocompatible polymer without a solubilizer, but no report is made on the change in properties according to the temperature of use, so there is no characteristic of thermosensitivity, and when injected into the skin of a laboratory rat (SD rat), a lump-like form can be confirmed in the tissue after 6 weeks, so it is not suitable for tissue repair.

In addition, since the particles of existing injectable agents for tissue repair are prepared with relatively high viscosity and elasticity by focusing on physical tissue repair, high injection force is required with injection needles of 30 gauge or larger, and 18 to 27 gauge injection needles are used to reduce inconveniences in the procedure such as needle clogging, but this causes great pain to the patient during the procedure.

In order to solve the above problems, the present inventors have invented a composition for tissue repair which can be easily used as an injectable agent by having a small particle size and a viscosity that decreases as the temperature increases. Based on the above, the present inventors have completed the present invention.

### PRIOR ART DOCUMENT

Patent Document

(Patent Document 0001) Korean Patent Application Publication No. 10-2020-0158100

(Patent Document 0002) Korean Patent Application Publication No. 10-2015-0053181

(Patent Document 0003) Korean Patent Application Publication No. 10-2016-0122111

(Patent Document 0004) Korean Patent Application Publication No. 10-2015-0010464

### DETAILED DESCRIPTION OF INVENTION

### Technical Problem

An object of the present invention is to provide a composition for tissue repair having excellent effects and physicochemical properties, the composition comprising a copolymer of a hydrophilic biocompatible polymer and a hydrophobic biocompatible polymer.

In addition, another object of the present invention is to provide a method for preparing the composition for tissue repair.

### Solution to Problem

The present invention provides a composition for tissue repair, the composition comprising a copolymer of a hydrophilic biocompatible polymer and a hydrophobic biocompatible polymer, wherein a weight ratio of the hydrophilic biocompatible polymer and the hydrophobic biocompatible polymer included in the copolymer of a hydrophilic biocompatible polymer and a hydrophobic biocompatible polymer is 1:1 to 15:1, and a molecular weight of the copolymer is 1,000 to 10,000 g/mol.

The weight ratio of the hydrophilic biocompatible polymer and the hydrophobic biocompatible polymer included in the copolymer of a hydrophilic biocompatible polymer and a hydrophobic biocompatible polymer may be preferably 2:1 to 8:1, and more preferably 2:1 to 4:1.

The molecular weight of the copolymer may be preferably 3,000 to 9,000 g/mol.

The concentration of the copolymer of a hydrophilic biocompatible polymer and a hydrophobic biocompatible polymer in the composition may be 1 to 50 wt %, and preferably 5 to 40 wt %.

The particle size of the copolymer of a hydrophilic biocompatible polymer and a hydrophobic biocompatible polymer may be 10 μm or less, preferably 5 μm or less, and more preferably 1 μm or less.

The composition for tissue repair, comprising a copolymer of a hydrophilic biocompatible polymer and a hydrophobic biocompatible polymer may have a viscosity that decreases as the temperature increases.

The hydrophilic biocompatible polymer may be at least one selected from the group consisting of methoxy polyethylene glycol, dihydroxy polyethylene glycol, monoalkoxy polyethylene glycol, monoacyl polyethylene glycol, and polyethylene glycol.

The hydrophobic biocompatible polymer may be at least one selected from the group consisting of polycaprolactone, polyglycolic acid, and a copolymer comprising the same.

The composition for tissue repair, comprising a copolymer of a hydrophilic biocompatible polymer and a hydrophobic biocompatible polymer may be an injectable formulation, and an injection force of the injectable formulation may be 15 N or less.

The composition for tissue repair, comprising a copolymer of a hydrophilic biocompatible polymer and a hydrophobic biocompatible polymer may be biodegraded within 12 weeks after intradermal injection, preferably within 10 weeks after intradermal injection, and more preferably within 2 to 8 weeks after intradermal injection.

The copolymer of a hydrophilic biocompatible polymer and a hydrophobic biocompatible polymer of the present invention may be preferably a methoxy polyethylene glycol-polycaprolactone copolymer.

### Effects of Invention

The composition of the present invention has excellent biocompatibility and collagen regeneration effects, so it can be used as a composition for tissue repair. In addition, the composition has excellent properties such as thermosensitivity in which the viscosity decreases as the temperature increases and injectability into the body, so that it can be used as an injectable formulation without concern for side effects.

### BRIEF DESCRIPTION OF DRAWINGS

FIG. 1 shows a methoxy polyethylene glycol-polycaprolactone copolymer (wherein n and m each represent an integer) and a method for calculating the polymerization ratio of a copolymer of a hydrophilic biocompatible polymer and a hydrophobic biocompatible polymer using nuclear magnetic resonance spectroscopy.

FIG. 2 shows whether the composition prepared according to Example 1 produces a precipitate.

FIG. 3 shows the properties according to the concentration of the composition prepared according to Example 5.

FIG. 4 shows the injectable agent of the composition comprising the methoxy polyethylene glycol-polycaprolactone copolymer of Preparation Example 5 prepared according to Examples 1 and 2.

FIG. 5 shows the skin changes over time after the composition comprising the methoxy polyethylene glycol-polycaprolactone copolymer of Preparation Example 5 prepared according to Examples 1 and 2 was injected into a laboratory rat. FIG. 6 shows the results of Hemato & Eosin staining of skin tissue 4 weeks after the composition comprising the methoxy polyethylene glycol-polycaprolactone copolymer of Preparation Example 5 prepared according to Examples 1 and 2 was injected into a laboratory rat.

FIG. 7 shows the results of Masson's Trichrome staining of skin tissue 4 weeks after the composition comprising the methoxy polyethylene glycol-polycaprolactone copolymer of Preparation Example 5 prepared according to Examples 1 and 2 was injected into a laboratory rat.

FIG. 8 shows the results obtained by measuring skin thickness after the composition comprising the methoxy polyethylene glycol-polycaprolactone copolymer of Preparation Example 5 prepared according to Examples 1 and 2 was injected into a laboratory rat.

FIG. 9 shows the results of Hemato & Eosin staining of skin tissue 1 week, 2 weeks, and 8 weeks after the composition comprising the methoxy polyethylene glycol-polycaprolactone copolymer of Preparation Example 5 prepared according to Example 1 was injected into a laboratory rat.

FIG. 10 shows the results of Hemato & Eosin staining of skin tissue 1 week, 2 weeks, and 8 weeks after the composition comprising the methoxy polyethylene glycol-polycaprolactone copolymer of Preparation Example 5 prepared according to Example 2 was injected into a laboratory rat.

FIG. 11 shows the results of Hemato & Eosin staining of skin tissue 1 week, 2 weeks, and 8 weeks after the polycaprolactone composition for tissue repair of Comparative Example 3 was injected into a laboratory rat.

### MODE FOR CARRYING OUT THE INVENTION

Hereinafter, with reference to the accompanying drawings, embodiments and examples of the present disclosure will be described in detail so that those of ordinary skill in the art to which the present invention belongs can easily practice the present invention. However, the present disclosure may be implemented in various forms and is not limited to the embodiments and examples described herein.

Throughout the present specification, when a certain part “includes” a certain component, it means that other components may be further included, rather than excluding other components, unless otherwise stated.

The present invention provides a composition for tissue repair, comprising a copolymer of a hydrophilic biocompatible polymer and a hydrophobic biocompatible polymer, wherein the composition has a viscosity that decreases as the temperature increases.

In the present invention, “tissue repair” refers to a mechanism for returning tissue to its original state when necrosis or defects occur in the tissue due to trauma, inflammation, aging, and the like, or for regenerating tissue for the purpose of improving wrinkles on the face or body, improving contours, forming volume in the tissue, or healing scars.

The composition for tissue repair may be an aqueous solution or a hydrogel.

In the present invention, the “hydrophilic biocompatible polymer” is a polymer having affinity and biocompatibility for a water-soluble solvent, and collectively refers to all biocompatible polymers useful to the human body that can be added to biologically active substances such as natural or artificially synthetic polymer substances, and is mainly used for the purpose of improving the solubility and biocompatibility of drugs, but is not limited thereto.

The hydrophilic biocompatible polymer includes methoxy polyethylene glycol, dihydroxy polyethylene glycol, monoalkoxy polyethylene glycol, monoacyl polyethylene glycol, and polyethylene glycol, and it may be preferably methoxy polyethylene glycol.

In the present invention, the “hydrophobic biocompatible polymer” is a polymer having affinity and biocompatibility for a non-water-soluble solvent, and is used for the purpose of preparing a non-water-soluble pharmaceutical vehicle or hydrophobic medical material, but is not limited thereto.

The hydrophobic biocompatible polymer includes polycaprolactone, polyglycolic acid, and a copolymer comprising the same, and it may be preferably polycaprolactone.

In the present invention, the methoxy polyethylene glycol-polycaprolactone copolymer refers to a copolymer having the following structure.

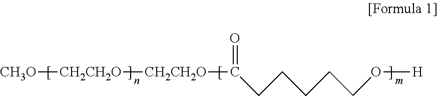

(wherein n and m each represent an integer.)

The methoxy polyethylene glycol-polycaprolactone copolymer refers to a linear amphiphilic copolymer formed by the association of mPEG-PCL, a polymerized polymer of methoxy polyethylene glycol, a hydrophilic substance, and polycaprolactone, a hydrophobic substance. In addition, the present invention provides a method for preparing the composition for tissue repair, comprising a copolymer of a hydrophilic biocompatible polymer and a hydrophobic biocompatible polymer.

The above method comprises a step of polymerizing the hydrophilic biocompatible polymer and the hydrophobic biocompatible polymer to prepare a copolymer, and a step of adding the copolymer to a physiological saline to obtain an aqueous solution.

Hereinafter, the present invention will be described in more detail through the examples, but the following examples are for illustrative purposes only and are not intended to limit the scope of the present invention.

### Preparation Example 1

### Methoxy Polyethylene Glycol-Polycaprolactone Copolymer 3,875 g/mol

A catalyst was added to 230 g of polyethylene glycol monomethyl ether (2,000 g/mol) and 114 g of monomer caprolactone (molecular weight: 114 g/mol), and polymerization was performed at 120° C. for 18 hours to prepare a methoxy polyethylene glycol-polycaprolactone copolymer having a molecular weight of 3,875 g/mol. The molecular weight was measured by GPC (gel chromatography).

### Preparation Example 2

### Methoxy Polyethylene Glycol-Polycaprolactone Copolymer 3,316 g/mol

A methoxy polyethylene glycol-polycaprolactone copolymer was prepared using 220 g of polyethylene glycol monomethyl ether (2,000 g/mol) and 72 g of monomer caprolactone in the same manner as in Preparation Example 1 above.

### Preparation Example 3

### Methoxy Polyethylene Glycol-Polycaprolactone Copolymer 3,349 g/mol

A methoxy polyethylene glycol-polycaprolactone copolymer was prepared using 290 g of polyethylene glycol monomethyl ether (2,000 g/mol) and 72 g of monomer caprolactone in the same manner as in Preparation Example 1 above.

### Preparation Example 4

### Methoxy Polyethylene Glycol-Polycaprolactone Copolymer 7,569 g/mol

A methoxy polyethylene glycol-polycaprolactone copolymer was prepared using 230 g of polyethylene glycol monomethyl ether (4,000 g/mol) and 114 g of monomer caprolactone in the same manner as in Preparation Example 1 above.

### Preparation Example 5

### Methoxy Polyethylene Glycol-Polycaprolactone Copolymer 8,464 g/mol

A methoxy polyethylene glycol-polycaprolactone copolymer was prepared using 220 g of polyethylene glycol monomethyl ether (4,000 g/mol) and 72 g of monomer caprolactone in the same manner as in Preparation Example 1 above.

### Preparation Example 6

### Methoxy Polyethylene Glycol-Polycaprolactone Copolymer 6,426 g/mol

A methoxy polyethylene glycol-polycaprolactone copolymer was prepared using 290 g of polyethylene glycol monomethyl ether (4,000 g/mol) and 72 g of monomer caprolactone in the same manner as in Preparation Example 1 above.

### Preparation Example 7

### Methoxy Polyethylene Glycol-Polycaprolactone Copolymer 7,721 g/mol

A methoxy polyethylene glycol-polycaprolactone copolymer was prepared using 230 g of polyethylene glycol monomethyl ether (5,000 g/mol) and 114 g of monomer caprolactone in the same manner as in Preparation Example 1 above.

### Preparation Example 8

### Methoxy Polyethylene Glycol-Polycaprolactone Copolymer 7,775 g/mol

A methoxy polyethylene glycol-polycaprolactone copolymer was prepared using 220 g of polyethylene glycol monomethyl ether (5,000 g/mol) and 72 g of monomer caprolactone in the same manner as in Preparation Example 1 above.

### Preparation Example 9

### Methoxy Polyethylene Glycol-Polycaprolactone Copolymer 7,882 g/mol

A methoxy polyethylene glycol-polycaprolactone copolymer was prepared using 290 g of polyethylene glycol monomethyl ether (5,000 g/mol) and 72 g of monomer caprolactone in the same manner as in Preparation Example 1 above.

### Preparation Example 10

### Methoxy Polyethylene Glycol-Polycaprolactone Copolymer 10,342 g/mol

A methoxy polyethylene glycol-polycaprolactone copolymer was prepared using 230 g of polyethylene glycol monomethyl ether (8,000 g/mol) and 114 g of monomer caprolactone in the same manner as in Preparation Example 1 above.

### Preparation Example 11

### Methoxy Polyethylene Glycol-Polycaprolactone Copolymer 9,776 g/mol

A methoxy polyethylene glycol-polycaprolactone copolymer was prepared using 220 g of polyethylene glycol monomethyl ether (8,000 g/mol) and 72 g of monomer caprolactone in the same manner as in Preparation Example 1 above.

### Preparation Example 12

### Methoxy Polyethylene Glycol-Polycaprolactone Copolymer 11,711 g/mol

A methoxy polyethylene glycol-polycaprolactone copolymer was prepared using 290 g of polyethylene glycol monomethyl ether (8,000 g/mol) and 72 g of monomer caprolactone in the same manner as in Preparation Example 1 above.

### Preparation Example 13

### Methoxy Polyethylene Glycol-Polycaprolactone Copolymer 847 g/mol

A methoxy polyethylene glycol-polycaprolactone copolymer was prepared using 230 g of polyethylene glycol monomethyl ether (400 g/mol) and 114 g of monomer caprolactone in the same manner as in Preparation Example 1 above.

### Comparative Example 1

### Cross-Linked Hyaluronic Acid Injection for Tissue Repair

It is a hydrogel formulation (1.1 mL) comprising 2.2% of cross-linked hyaluronic acid, and is an injection for tissue repair filled in a prefilled syringe (Product name: The Chaeum Premium No. 1, Manufacturer: Across Co., Ltd.).

### Comparative Example 2

### Cross-Linked Hyaluronic Acid Injection for Tissue Repair

It is a hydrogel formulation (1.1 mL) comprising 2.2% of cross-linked hyaluronic acid, and is an injection for tissue repair filled in a prefilled syringe (Product name: The Chaeum Premium No. 2, Manufacturer: Across Co., Ltd.).

### Comparative Example 3

### Polycaprolactone Injection for Tissue Repair

It is a hydrogel formulation (1 mL) comprising 25% of polycaprolactone and 75% of carboxymethyl cellulose, and is an injection for tissue repair filled in a prefilled syringe (Product name: Ellansé, Manufacturer: Sinclair) [symbol] .

### Example 1

### Composition for Tissue Repair Comprising Methoxy Polyethylene Glycol-Polycaprolactone Copolymer

Phosphate buffered physiological saline was added so that the concentration of each of the methoxy polyethylene glycol-polycaprolactone copolymers of Preparation Examples 1 to 13 above was 20%, and then mixed and stirred to prepare an aqueous solution.

### Example 2

### Composition for Tissue Repair Comprising Methoxy Polyethylene Glycol-Polycaprolactone Copolymer

Phosphate buffered physiological saline was added so that the concentration of each of the methoxy polyethylene glycol-polycaprolactone copolymers of Preparation Examples 1 to 12 above was 30%, and then mixed and stirred to prepare an aqueous solution.

### Example 3

### Composition for Tissue Repair Comprising Methoxy Polyethylene Glycol-Polycaprolactone Copolymer

Phosphate buffered physiological saline was added so that the concentration of each of the methoxy polyethylene glycol-polycaprolactone copolymers of Preparation Examples 1 to 12 above was 40%, and then mixed and stirred to prepare an aqueous solution.

### Example 4

### Composition for Tissue Repair Comprising Methoxy Polyethylene Glycol-Polycaprolactone Copolymer

Phosphate buffered physiological saline was added so that the concentration of each of the methoxy polyethylene glycol-polycaprolactone copolymers of Preparation Examples 1, 4, 5, and 6 above was 10%, and then mixed and stirred to prepare an aqueous solution.

### Example 5

### Composition for Tissue Repair Comprising Methoxy Polyethylene Glycol-Polycaprolactone Copolymer

Phosphate buffered physiological saline was added so that the concentration of each of the methoxy polyethylene glycol-polycaprolactone copolymers of Preparation Examples 1, 4, 5, and 6 above was 5%, 10%, 20%, 25%, 30%, and 40%, and then mixed and stirred to prepare an aqueous solution.

### Experimental Example 1

### Evaluation of Properties of Composition According to Polymerization Ratio and Molecular Weight of Copolymer of Hydrophilic Biocompatible Polymer and Hydrophobic Biocompatible Polymer

The compositions for tissue repair prepared according to Examples 1 and 2 above were bottled in glass vials (3 mL), sealed, and stored in a thermostat at 25° C. for 7 days. Thereafter, the presence or absence of precipitate was visually determined, and the compositions in which precipitate was observed were judged to be suspensions.

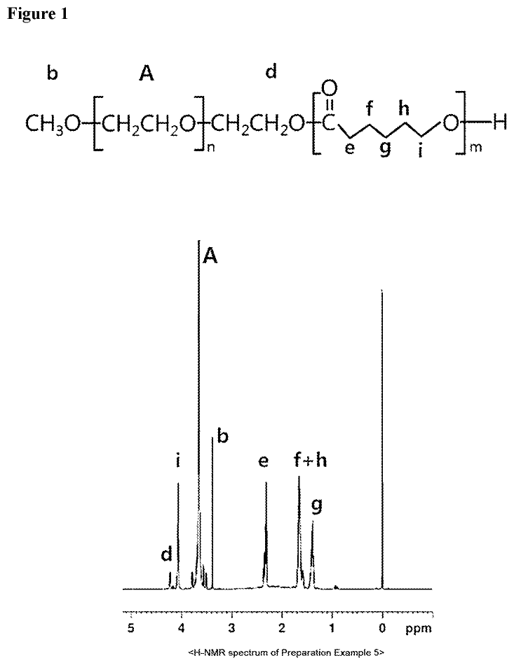

The molecular structure of the polymerized copolymer was analyzed through ¹H-NMR, and as shown in FIG. 1, each signal was checked to analyze the molecular structure, and then the polymerization ratio of the molecular weight of the hydrophilic polymer and the molecular weight of the hydrophobic polymer was calculated using the integral value of the peak.

TABLE 1

| | Polymerization | | | | | |
|---|---|---|---|---|---|---|
| | ratio of hydro- | Molecular | | | Precipitate | |
| | philic:hydro- | weight | Properties | | (formed ∘, none —) | |
| Item | phobic polymer | (g/mol) | Example 1 | Example 2 | Example 1 | Example 2 |
| Preparation Example 1 | 3.37:1 | 3,875 | sol | gel | — | — |
| Preparation Example 4 | 2.16:1 | 7,569 | gel | gel | — | — |
| Preparation Example 5 | 3.16:1 | 8,464 | sol | gel | — | — |
| Preparation Example 6 | 7.02:1 | 6,426 | sol | sol | — | — |
| Preparation Example 7 | 16.4:1 | 7,721 | suspension | suspension | ∘ | ∘ |
| Preparation Example 10 | 8.14:1 | 10,342 | suspension | suspension | ∘ | ∘ |
| Preparation Example 11 | 23.15:1 | 9,776 | suspension | suspension | ∘ | ∘ |
| Preparation Example 12 | 27.8:1 | 11,711 | suspension | suspension | ∘ | ∘ |

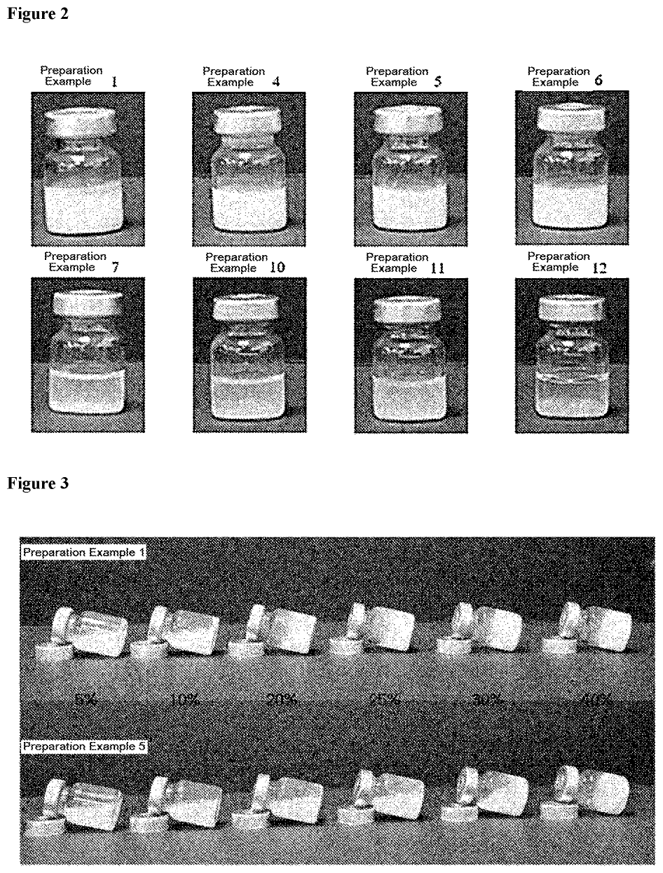

As shown in Table 1 above and FIG. 2, among the compositions prepared according to Examples 1 and 2, the compositions comprising the methoxy polyethylene glycol-polycaprolactone copolymers of Preparation Examples 1, 4, 5, and 6 did not produce a precipitate and also showed significantly small average particle sizes.

Therefore, it can be seen that a copolymer having a polymerization ratio of a copolymer of a hydrophilic biocompatible polymer and a hydrophobic biocompatible polymer of 1:1 to 15:1 and a molecular weight of 1,000 to 10,000 g/mol has excellent physicochemical characteristics and properties.

### Experimental Example 2

### Evaluation of Properties of Composition According to Concentration of Copolymer of Hydrophilic Biocompatible Polymer and Hydrophobic Biocompatible Polymer

The properties of the composition for tissue repair comprising the methoxy polyethylene glycol-polycaprolactone copolymers of Preparation Examples 1, 4, 5, and 6 prepared according to Example 5 above were compared according to the concentration, and the results are shown in Table 2 below and FIG. 3.

TABLE 2

| | Properties by concentration | | | | | |
|---|---|---|---|---|---|---|
| Item | 5% | 10% | 20% | 25% | 30% | 40% |
| Preparation Example 1 | sol | sol | sol | gel | gel | gel |
| Preparation Example 4 | sol | sol | gel | gel | gel | gel |
| Preparation Example 5 | sol | sol | sol | sol | sol | gel |
| Preparation Example 6 | sol | sol | sol | gel | gel | gel |

As shown in Table 2 above and FIG. 3, it was confirmed that all of the compositions comprising the methoxy polyethylene glycol-polycaprolactone copolymers of Preparation Examples 1, 4, 5, and 6 at a concentration of 5 to 40% maintained a sol or gel form.

Therefore, it can be seen that the composition of the present invention has excellent properties that can be injected into the body when the copolymer of a hydrophilic biocompatible polymer and a hydrophobic biocompatible polymer is included at a concentration of 5 to 40%.

### Experimental Example 3

### Evaluation of Viscosity According to Temperature Change of Composition Comprising Copolymer of Hydrophilic Biocompatible Polymer and Hydrophobic Biocompatible Polymer

The compositions for tissue repair comprising the methoxy polyethylene glycol-polycaprolactone copolymer of Preparation Examples 1, 4, and 5 prepared according to Examples 1 and 3 above were filled into a glass container and immersed in a constant temperature water bath set to a temperature of 5° C., 20° C., 25° C., 30° C., and 35° C., respectively, for 20 minutes, and then the viscosity was measured using a viscometer (VISCO B, ATAGO). The results are shown in Table 3 below.

TABLE 3

| | | Viscosity by temperature (mPa · s) | | | | |
|---|---|---|---|---|---|---|
| Item | | 5° C. | 20° C. | 25° C. | 30° C. | 35° C. |
| Example 1 | Preparation Example 1 | 449 | 213 | 180 | 84 | 31 |
| | Preparation Example 4 | 36,102 | 10,605 | 9,319 | 1,202 | 948 |
| | Preparation Example 5 | 279 | 179 | 160 | 97 | 43 |
| Example 3 | Preparation Example 1 | 28,565 | 9,735 | 3,152 | 1,238 | 735 |
| | Preparation Example 5 | 15,605 | 3,089 | 2,328 | 968 | 427 |

As shown in Table 3 above, it was confirmed that the composition comprising the methoxy polyethylene glycol-polycaprolactone copolymer of Preparation Examples 1, 4, and 5 prepared according to Examples 1 and 3 had a viscosity that decreased as the temperature increased.

Therefore, it can be seen that the composition comprising the copolymer of a hydrophilic biocompatible polymer and a hydrophobic biocompatible polymer of the present invention has excellent properties that can be injected into the body.

### Experimental Example 4

### Evaluation of Injection Force of Injection Needle of Composition Comprising Copolymer of Hydrophilic Biocompatible Polymer and Hydrophobic Biocompatible Polymer

After storing in a thermostat maintained at a temperature of 25° C. for 48 hours or more, the compositions for tissue repair comprising the methoxy polyethylene glycol-polycaprolactone copolymers of Preparation Examples 1, 4, 5, and 6 prepared according to Examples 1 and 2 above were filled into a prefilled syringe, and then a 26G injection needle and a 30G injection needle were each tied to check the injection force. The injection force was measured by pushing the plunger at a speed of 12 mm/min using a compression tester (MCT-1150, AND). In Comparative Examples 1 to 3, the injection force was checked by tying a 26G injection needle and a 30G injection needle to the existing product, respectively. The results are shown in Table 4 below.

TABLE 4

| | | | Injection force (N) | |
|---|---|---|---|---|
| Item | | Properties | 26G | 30G |
| Example 1 | Preparation Example 1 | gel | 1.2 | 4.1 |
| | Preparation Example 4 | gel | 3.9 | 14.5 |
| | Preparation Example 5 | sol | 1.6 | 4.8 |
| | Preparation Example 6 | sol | 0.9 | 2 |
| Example 2 | Preparation Example 1 | gel | 2.6 | 10.2 |
| | Preparation Example 5 | gel | 2.7 | 8.8 |
| | Preparation Example 6 | gel | 1.3 | 4.8 |
| Comparative | Comparative Example 1 | gel | 16 | 37.2 |
| Example | Comparative Example 2 | gel | 10.9 | 23 |
| | Comparative Example 3 | gel | 23 | 34 |

As shown in Table 4 above, it was confirmed that the compositions comprising the methoxy polyethylene glycol-polycaprolactone copolymers of Preparation Examples 1, 4, 5, and 6 prepared according to Examples 1 and 2 showed an injection force of 15 N or less in both a 26G injection needle and a 30G injection needle, which was significantly lower than the injection force of the compositions of Comparative Examples 1 to 3.

Therefore, it can be seen that the composition comprising the copolymer of a hydrophilic biocompatible polymer and a hydrophobic biocompatible polymer of the present invention has excellent properties that can be injected into the body.

### Experimental Example 5

### Analysis of Particle Size of Composition Comprising Copolymer of Hydrophilic Biocompatible Polymer and Hydrophobic Biocompatible Polymer

In order to measure the particle size of the compositions prepared according to Examples 1 and 2 above, they were divided into a preparation example in which a precipitate was formed and a group in which a sol or gel was formed without a precipitate being formed. The particle sizes of the compositions comprising the methoxy polyethylene glycol-polycaprolactone copolymer of Preparation Examples 7, 10, 11, and 12 prepared according to Example 1 in which a precipitate is formed were measured by laser diffraction and scattering using Mastersizer 2000 (Manufacturer: MALVERN). The particle sizes of the compositions comprising the methoxy polyethylene glycol-polycaprolactone copolymers of Preparation Examples 1, 4, 5, and 6 prepared according to Examples 1 and 2 in the form of sol and gel in which no precipitate is formed were measured by dynamic light scattering using ELSZ-1000 (Manufacturer: OTSUKA ELECTRONICS). The results are shown in Table 5 below.

TABLE 5

| | | | Average particle |
|---|---|---|---|
| | Item | Properties | size (μm) |
| Example 1 | Preparation Example 1 | gel | 1.0 |
| | Preparation Example 4 | gel | 0.3 |
| | Preparation Example 5 | sol | 0.7 |
| | Preparation Example 6 | sol | 0.8 |
| | Preparation Example 7 | suspension | 14.1 |
| | Preparation Example 10 | suspension | 21.6 |
| | Preparation Example 11 | suspension | 33.0 |
| | Preparation Example 12 | suspension | 25.8 |
| Example 2 | Preparation Example 1 | gel | 0.8 |
| | Preparation Example 5 | gel | 0.4 |
| Comparative | Comparative Example 1 | gel | not measured |
| Example | Comparative Example 2 | gel | not measured |
| | Comparative Example 3 | gel | 42 (reported value*) |

*Kim J. (2020). Isovolemic Degradation of Polycaprolactone Particles and Calculation of Their Original Size from Human Biopsy. Plastic and reconstructive surgery. Global open, 8 (6), e2866.

As shown in Table 5 above, it was confirmed that the compositions comprising the methoxy polyethylene glycol-polycaprolactone copolymers of Preparation Examples 1, 4, 5, and 6 prepared according to Examples 1 and 2 in the form of sol and gel in which no precipitate is formed had small particle sizes, and it was confirmed that the compositions had significantly smaller sizes compared to the particle sizes of Comparative Example 3.

Therefore, it can be seen that the composition comprising the copolymer of a hydrophilic biocompatible polymer and a hydrophobic biocompatible polymer of the present invention has excellent properties that can be injected into the body.

### Experimental Example 6

### Evaluation of Tissue Repair Ability of Composition Comprising Copolymer of Hydrophilic Biocompatible Polymer and Hydrophobic Biocompatible Polymer

In order to evaluate the biocompatibility and confirm collagen regeneration through intradermal transplantation of the composition for tissue repair of the present invention, the following experiment was conducted.

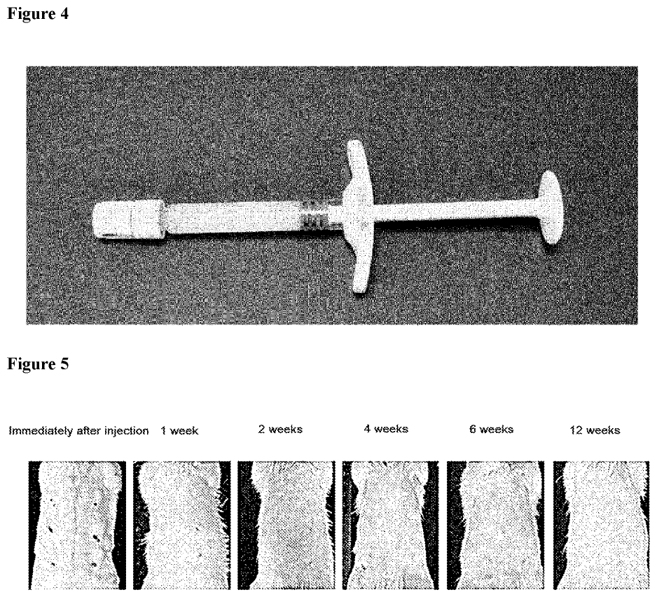

In the experimental group, the composition comprising the methoxy polyethylene glycol-polycaprolactone copolymer of Preparation Example 5 prepared according to Examples 1 and 2 above was filled into a 1 cc syringe and a 30 gauge injection needle was used (see FIG. 4). In the control group, 0.2 mL of physiological saline was filled into a 1 cc syringe and a 30 gauge injection needle was used.

0.2 mL of the composition comprising the methoxy polyethylene glycol-polycaprolactone copolymer of Preparation Example 5 prepared according to Examples 1 and 2 above was injected intradermally into the cervicodorsal region of a laboratory rat (SD rat). After injection, the SD rats were sacrificed every 0, 1, 2, 4, 6, and 12 weeks, and then the skin at the injection site was incised to extract skin tissue. The skin tissue was stained with Hemato & Eosin and Masson's Trichrome to determine histopathological evaluation, skin thickness, collagen regeneration, and the like. Histopathological evaluation of the injection site was graded based on the histologic score, as shown in Table 6 below, and skin thickness was measured using cellsens software (Olympus) after photographing the stained skin tissue with an optical microscope (BX51, Olympus) and a digital camera (DP74, Olympus). Only the epidermis and dermis were included in the measurement.

TABLE 6

| Grade | | Histologic findings |
|---|---|---|
| 0 | No | No visible reaction |
| | inflammation | |
| 1 | Almost clear | Slight reaction with a few inflammatory cells |
| 2 | Mild | Clear inflammatory reaction with one or two giant |
| | | cells |
| 3 | Moderate | Fibrous tissue with inflammatory cells, lymphocytes, |
| | | and giant cells |
| 4 | Severe | Granuloma with encapsulated implant-clear foreign |
| | | body reaction |

As a result, the H&E stained slides of the experimental and control groups were evaluated histopathologically, and it was confirmed that there was no foreign body reaction during the entire test period, which was determined to be grade 0 or 1.

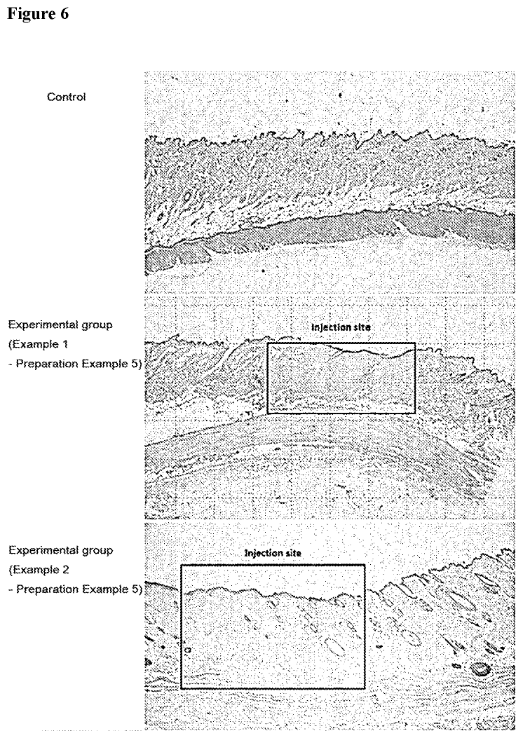

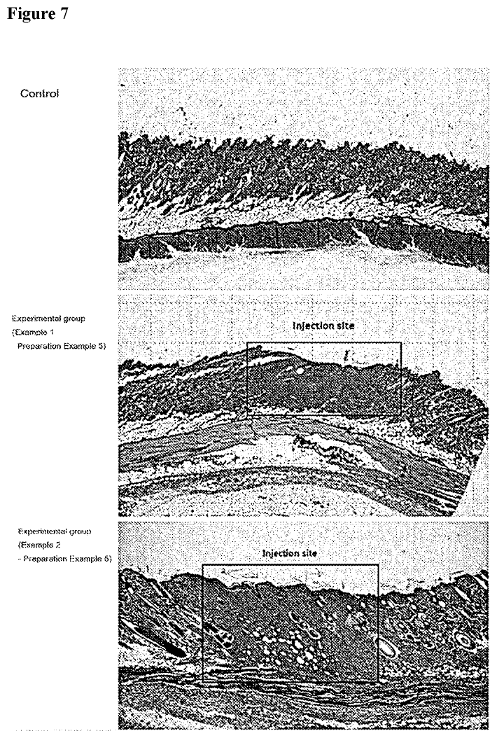

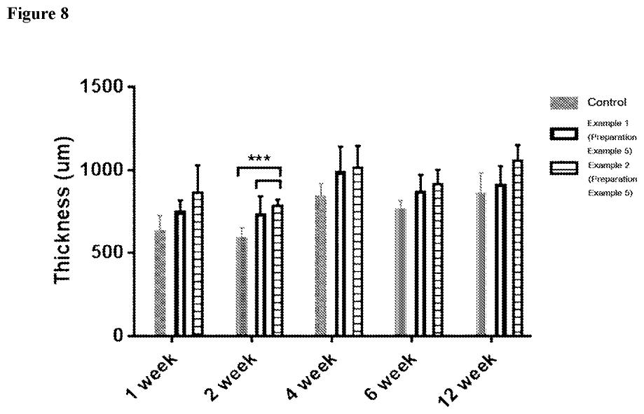

In addition, as shown in FIGS. 5 to 8, it was confirmed that the skin area injected with the composition comprising the methoxy polyethylene glycol-polycaprolactone copolymer of Preparation Example 5 prepared according to Examples 1 and 2 showed an increase in the thickness of the skin and the density of the dermis compared to normal tissue, and collagen regeneration was also confirmed based on this.

### Experimental Example 7

### Evaluation of Biodegradability of Composition Comprising Copolymer of Hydrophilic Biocompatible Polymer and Hydrophobic Biocompatible Polymer

In order to confirm the biodegradation time of the composition for tissue repair of the present invention through intradermal transplantation, the following experiment was conducted.

In the experimental group, as in Experimental Example 6 above, the composition comprising the methoxy polyethylene glycol-polycaprolactone copolymer of Preparation Example 5 prepared according to Examples 1 and 2 above was filled into a 1 cc syringe and a 30 gauge injection needle was used. In the control group, a 27 gauge injection needle was used for Comparative Example 3 (Product name: Ellansé).

0.2 mL of the above experimental group and control group were each injected intradermally into the cervicodorsal region of the laboratory rat (SD rat), and the SD rats were sacrificed every 2, 4, 6, and 8 weeks from the injection time, and then the skin at the injection site was incised to extract skin tissue. The skin tissue was stained with Hemato & Eosin, and the stained skin tissue was photographed with an optical microscope (BX51, Olympus) and a digital camera (DP74, Olympus). The presence or absence of particles of the composition for tissue repair remaining at the injection site and the foreign body reaction were examined to confirm biodegradability.

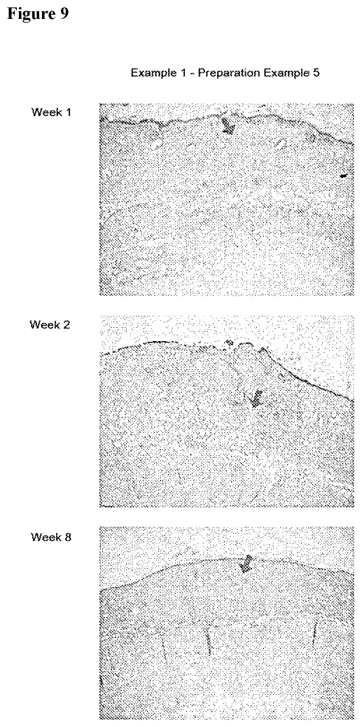

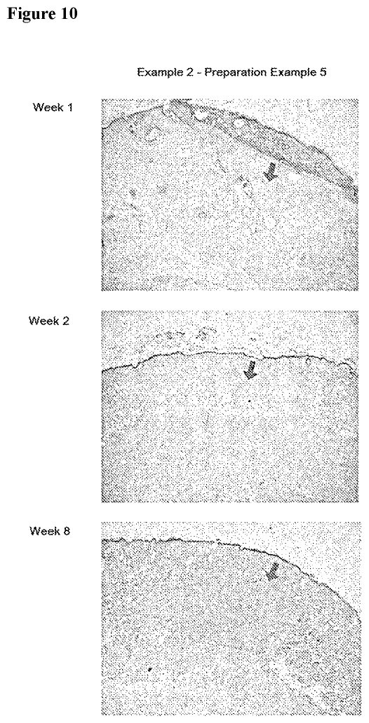

As a result, as shown in FIGS. 9 and 10, the foreign body reaction of the composition for tissue repair observed at 1 and 2 weeks after injecting into the skin area the composition comprising the methoxy polyethylene glycol-polycaprolactone copolymer of Preparation Example 5 prepared according to Examples 1 and 2 was not confirmed after 8 weeks, and no particles of the composition for tissue repair were confirmed.

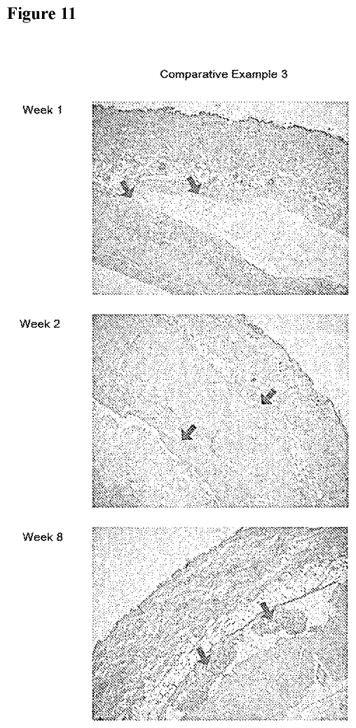

On the other hand, in the case of the polycaprolactone-based composition for tissue repair of Comparative Example 3, the foreign body reaction observed at 1 and 2 weeks was confirmed up to 8 weeks, and particles of the composition for tissue repair were also continuously confirmed (FIG. 11).

Therefore, it can be seen that the composition of the present invention has excellent biodegradability in the body.

1. A composition for tissue repair, comprising a copolymer of a hydrophilic biocompatible polymer and a hydrophobic biocompatible polymer, wherein a weight ratio of the hydrophilic biocompatible polymer and the hydrophobic biocompatible polymer included in the copolymer of a hydrophilic biocompatible polymer and a hydrophobic biocompatible polymer is 1:1 to 15:1, and a molecular weight of the copolymer is 1,000 to 10,000 g/mol.

2. The composition for tissue repair according to claim 1, wherein the weight ratio of the hydrophilic biocompatible polymer and the hydrophobic biocompatible polymer included in the copolymer of a hydrophilic biocompatible polymer and a hydrophobic biocompatible polymer is 2:1 to 8:1.

3. The composition for tissue repair according to claim 1, wherein the molecular weight of the copolymer is 3,000 to 9,000 g/mol.

4. The composition for tissue repair according to claim 1, wherein the weight ratio of the hydrophilic biocompatible polymer and the hydrophobic biocompatible polymer included in the copolymer of a hydrophilic biocompatible polymer and a hydrophobic biocompatible polymer is 2:1 to 4:1.

5. The composition for tissue repair according to claim 1, wherein a concentration of the copolymer of a hydrophilic biocompatible polymer and a hydrophobic biocompatible polymer in the composition is 1 to 50 wt %.

6. The composition for tissue repair according to claim 1, wherein a concentration of the copolymer of a hydrophilic biocompatible polymer and a hydrophobic biocompatible polymer in the composition is 5 to 40 wt %.

7. The composition for tissue repair according to claim 1, wherein a particle size of the copolymer of a hydrophilic biocompatible polymer and a hydrophobic biocompatible polymer is 10 μm or less.

8. The composition for tissue repair according to claim 1, wherein a particle size of the copolymer of a hydrophilic biocompatible polymer and a hydrophobic biocompatible polymer is 5 μm or less.

9. The composition for tissue repair according to claim 1, wherein a particle size of the copolymer of a hydrophilic biocompatible polymer and a hydrophobic biocompatible polymer is 1 μm or less.

10. The composition for tissue repair according to claim 1, wherein the composition has a viscosity that decreases as the temperature increases.

11. The composition for tissue repair according to claim 1, wherein the hydrophilic biocompatible polymer is at least one selected from the group consisting of methoxy polyethylene glycol, dihydroxy polyethylene glycol, monoalkoxy polyethylene glycol, monoacyl polyethylene glycol, and polyethylene glycol.

12. The composition for tissue repair according to claim 1, wherein the hydrophobic biocompatible polymer is at least one selected from the group consisting of polycaprolactone, polyglycolic acid, and a copolymer comprising the same.

13. The composition for tissue repair according to claim 1, wherein the composition is an injectable formulation.

14. The composition for tissue repair according to claim 13, wherein an injection force of the injectable formulation is 15 N or less.

15. The composition for tissue repair according to claim 13, wherein the composition is biodegraded within 12 weeks after intradermal injection.

16. The composition for tissue repair according to claim 13, wherein the composition is biodegraded within 10 weeks after intradermal injection.

17. The composition for tissue repair according to claim 13, wherein the composition is biodegraded within 2 to 8 weeks after intradermal injection.

18. The composition for tissue repair according to claim 1, wherein the copolymer of a hydrophilic biocompatible polymer and a hydrophobic biocompatible polymer is a methoxy polyethylene glycol-polycaprolactone copolymer.
